(19) United States
(12) Patent Application Publication
(10) Pub. No.: US 2021/0330886 A1
(43) Pub. Date: Oct. 28, 2021

(54) SAFETY SYRINGE

(71) Applicant: Chih-Hua HUNG, Hsinchu City (TW)

(72) Inventor: Chih-Hua HUNG, Hsinchu City (TW)

(21) Appl. No.: 16/631,036

(22) PCT Filed: Jul. 14, 2017
(86) PCT No.: PCT/CN2017/092891

§ 371 Date: Feb. 14, 2020

(51) Int. Cl. A61M 5/315; A61M 5/31; A61M 5/19

(52) U.S. Cl. CPC A61M 5/31505; A61M 5/3134; A61M 2005/31506; A61M 5/19; A61M 2005/3131; A61M 5/3158

(57) ABSTRACT

A safety syringe includes an outer barrel having a receiving groove located on an inner surface of a rear end thereof between a first annular flange and a second annular flange, an inner barrel having a needle mount located on a front end thereof and a positioning portion adjacent to the needle mount, and a plunger axially movably inserted into the inside of the inner barrel.

### BACKGROUND OF THE INVENTION

### 1. Field of the Invention

The present invention relates to safety syringes and more particularly, to such a safety syringe that has a simple structure and is easy to assemble.

### 2. Description of the Related Art

A syringe is a device to be used with a needle for injecting liquids such as medical fluid, blood or nutritional fluid into the human body. After the injection is completed, the needle is already contaminated with human blood, so the used needle must be safely processed to avoid medical staff or other people being injured by the needle. The early safe handling method was to insert the used needle into a needle cover for storage, but during the insertion process, the hands of the medical personnel may be accidentally pierced by the needle due to their carelessness or being pushed by other external forces, which in turn increases the risk of accidental infections for the medical personnel. Such problems also lead to so-called safety syringes to reduce the chance of medical personnel directly touching the needle with their hands.

As for the traditional safety syringe, after the medical personnel inserts the plunger into the outer barrel to complete the injection, the plunger will be locked with the needle holder through a special structural design, so that the needle holder with the needle can be pulled back into the outer barrel by the plunger. However, in the aforementioned prior art, the medical personnel needs to apply a large thrust to the plunger in order to form a snap connection between the plunger and the needle holder. This will not only cause medical personnel's assembly trouble, but if the force is too large, it may cause structural damage. Therefore, it is necessary to improve the structure of traditional safety syringes.

### SUMMARY OF THE INVENTION

The present invention has been accomplished under the circumstances in view. It is the main object of the present invention to provide a safety syringe, which has the characteristics of simple structure and easy assembly to increase convenience in use.

To achieve this and other objects of the present invention, a safety syringe comprises an outer barrel, an inner barrel, and a plunger. The outer barrel comprises a front end opening located in a front end thereof, a first rear end opening located in an opposing rear end thereof, a first annular flange and a second annular flange located on an inner surface of the rear end of the outer barrel, and a receiving groove located on the inner surface of the rear end of the outer barrel between the first annular flange and the second annular flange. The inner barrel comprises a needle mount located on a front end thereof, a positioning portion adjacent to the needle mount, a second rear end opening located in an opposing rear end thereof. The plunger is inserted through the second rear end opening of the inner barrel into the inside of the inner barrel and axially movable relative to the inner barrel. The inner barrel is inserted through the first rear end opening of the outer barrel into the inside of the outer barrel and axially movable relative to the outer barrel between a first position and a second position. When the inner barrel is located at the first position, the positioning portion of the inner barrel is located between the front end opening of the outer barrel and the second annular flange of the outer barrel so that the needle mount of the inner barrel is partially exposed to the outside of the outer barrel through the front end opening. When the inner barrel is located at the second position, the positioning portion of the inner barrel is located in the receiving groove of the outer barrel and blocked by the first annular flange and second annular flange of the outer barrel so that the needle mount of the inner barrel is completely inside the outer barrel.

It can be known from the above, when the inner barrel is located at the first position, the positioning portion of the inner barrel is located between the front end opening and second annular flange of the outer barrel, so that the inner barrel can be forced by an external force to move the needle mount partially out of the outer barrel through the front end opening, letting a needle in the needle mount of the inner barrel make an injection.

When the inner barrel is moved from the first position to the second position, the positioning portion of the inner barrel is located within the receiving groove of the outer barrel, and, the positioning portion of the inner barrel is blocked by the first annular flange and second annular flange of the outer barrel, so that the range of movement of the inner barrel is limited between the first annular flange and second annular flange of the outer barrel. At this time, the needle mount of the inner barrel and the needle are kept inside the outer barrel to increase safety after injection.

Preferably, the inner diameter of the second annular flange of the outer barrel gradually increases in direction away from the receiving groove of the outer barrel, so that when the inner barrel is being moved from the first position to the second position, the positioning portion can be moved over the second annular flange of the outer barrel with less effort.

Preferably, the first rear end opening of the outer barrel is adjacent to the first annular flange of the outer barrel, and the inner diameter of the first rear end opening of the outer barrel gradually increases in direction away from the first annular flange of the outer barrel, so that when the inner barrel is assembled in the outer barrel, the positioning portion can be moved over the first annular flange of the outer barrel with less effort.

Preferably, the axial length of the receiving groove of the outer barrel is larger than the axial length of the positioning portion of the inner barrel. When the inner barrel is located at the second position, a force can be applied to make the outer barrel skew so that the front end opening of the outer barrel and the needle mount of the inner barrel are staggered from each other and not on the same line. In this case, even the inner barrel or the plunger accidental collision, the needle will not extend through the front end opening of the outer barrel to the outside of the outer barrel, to further ensure that the needle has sufficient safety after injection.

Preferably, the positioning portion of the inner barrel is composed of four bumps equiangularly spaced around the central axis of the inner barrel. Alternatively, the positioning portion of the inner barrel can be an annular bump.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a sectional exploded plain view of a safety syringe in accordance with a first embodiment of the present invention.

FIG. 2 is a sectional plain assembly view of the inner and outer barrels of the safety syringe in accordance with the first embodiment of the present invention, mainly illustrating the inner barrel in the second position.

FIG. 3 is similar to FIG. 2, mainly illustrating the inner barrel in the first position.

FIG. 4 is a top view of the inner barrel of the safety syringe in accordance with the first embodiment of the present invention.

FIG. 5 is a sectional assembly plain view of the safety syringe in accordance with the first embodiment of the present invention, mainly illustrating the needle protruded over the outer barrel.

FIG. 6 is similar to FIG. 5, illustrating the needle received inside the outer barrel.

FIG. 7 is similar to FIG. 6, illustrating the outer barrel skewed relative to the inner barrel.

FIG. 8 is a schematic sectional exploded view of a part of a safety syringe in accordance with a second embodiment of the present invention, illustrating an alternate form of the needle mount.

FIG. 9 is a schematic sectional exploded view of a part of a safety syringe in accordance with a third embodiment of the present invention, illustrating an alternate form of the positioning portion.

FIG. 10 is a top view of FIG. 9.

### DETAILED DESCRIPTION OF THE INVENTION

The following describes the technical content and features of the present invention in detail with the enumerated several preferred embodiments and the drawings. The directional adjectives of “front”, “rear”, “inside”, “outside” and the like mentioned in the description of this specification are merely examples of descriptions based on the direction of normal use and are not intended to limit the scope of claims.

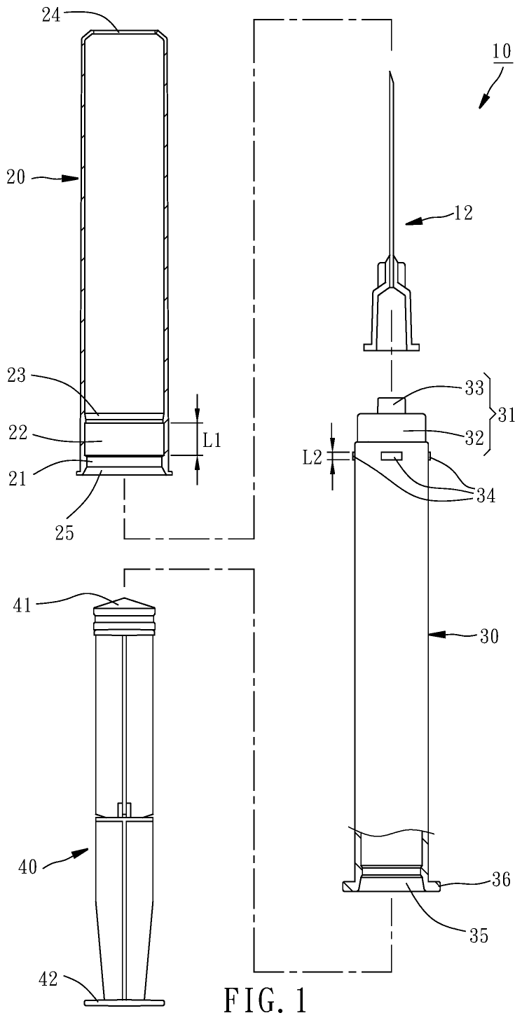

Referring to FIG. 1, a safety syringe 10 in accordance with a first embodiment of the present invention comprises an outer barrel 20, an inner barrel 30, and a plunger 40.

The outer barrel 20 is made of plastic. In order from back to front, the inner wall surface of the rear end of the outer barrel 20 has a first annular flange 21, a receiving groove 22 adjacent to the first annular flange 21, and a second annular flange 23 adjacent to the receiving groove 22. The second annular flange 23 gradually increases in the inner diameter in direction away from the receiving groove 22. In addition, the outer barrel 20 has a front end opening 24 and a first rear end opening 25. The first rear end opening 25 is adjacent to the first annular flange 21, and the first rear end opening 25 gradually increases in the inner diameter in direction away from the first annular flange 21.

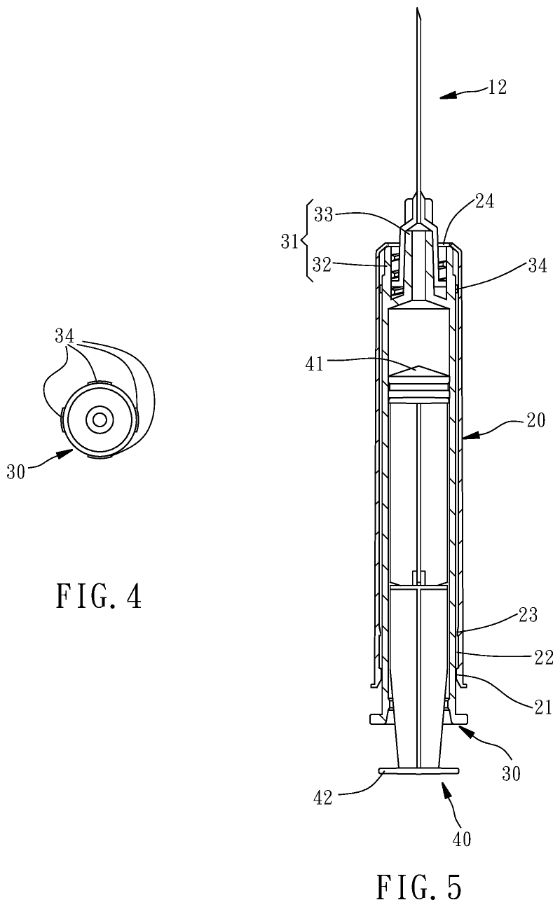

The inner barrel 30 is made of plastic. The front end of the inner barrel 30 has a needle mount 31 and a positioning portion 34 adjacent to the needle mount 31. The needle mount 31 in this embodiment is composed of a screwing base 32 and an adapter shaft 33 located on the screwing base 32. Therefore, when used with the needle 12, the needle 12 is screwed to the screwing base 32 of the needle mount 31 of the inner barrel 30 and simultaneously attached onto the adapter shaft 33 of the needle mount 31 of the inner barrel 30. As shown in FIG. 4, the positioning portion 34 in this embodiment is composed of four bumps that are equiangularly spaced around the central axis of the inner barrel 30. The axial length L2 of the positioning portion 34 is shorter than the axial length L1 of the receiving groove 22 of the outer barrel 20. In addition, the rear end of the inner barrel 30 has a second rear end opening 35 and a finger flange 36 outwardly extended from the periphery of the second rear end opening 35.

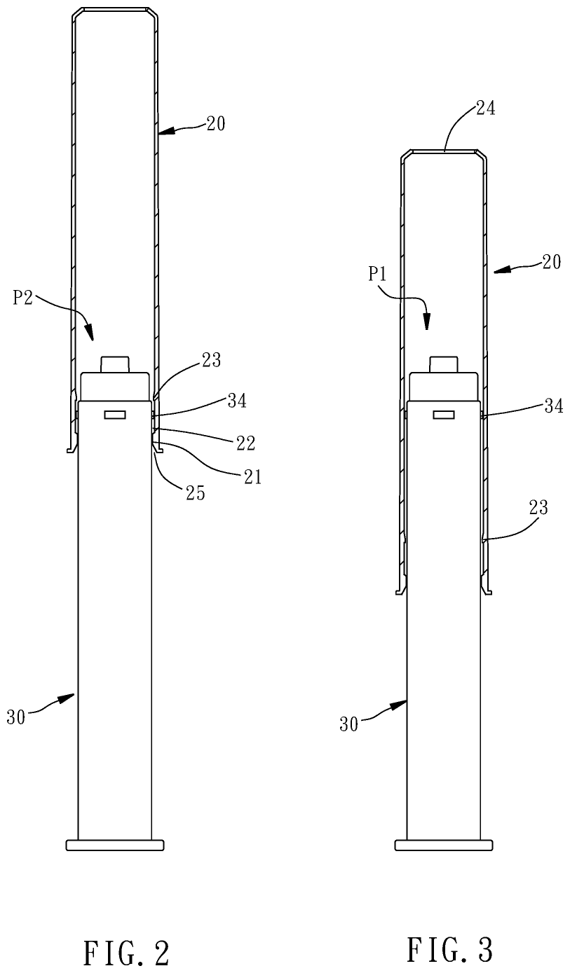

When the inner barrel 30 is assembled in the outer barrel 20, the inner diameter of the first rear end opening 25 gradually increases in direction away from the first annular flange 21, so the inner barrel 30 can easily be inserted through the first rear end opening 25 of the outer barrel 20 into the inside of the outer barrel 20. During the process of inserting the inner barrel 30 into the outer barrel 20, the positioning portion 34 of the inner barrel 30 will be squeezed to deform by the first annular flange 21 of the outer barrel 20. Because the protruding height of the positioning portion 34 of the inner barrel 30 is limited, the positioning portion 34 of the inner barrel 30 can be moved over the first annular flange 21 of the outer barrel 20 into the receiving groove 22 of the outer barrel 20 without applying a large thrust to the inner barrel 30. The inner barrel 30 at this time is located at a second position P2 (see FIG. 2). When the inner barrel 30 is located at the second position P2 as shown in FIG. 2, the positioning portion 34 of the inner barrel 30 is located within the receiving groove 22 of the outer barrel 20, and the positioning portion 34 of the inner barrel 30 is blocked by the first annular flange 21 and second annular flange 23 of the outer barrel 20, so that the movement range of the inner barrel 30 at this time is limited between the first annular flange 21 and second annular flange 23 of the outer barrel 20. Then, continue to apply thrust to the inner barrel 30 to let the positioning portion 34 of the inner barrel 30 pass the second annular flange 23 of the outer barrel 20 to complete the assembly of the two. At this time, the inner barrel 30 is located at a first position P1 (see FIG. 3). When the inner barrel 30 is located at the first position P1 as shown in FIG. 3, the positioning portion 34 of the inner barrel 30 is located between the front end opening 24 of the outer barrel 20 and the second annular flange 23 of the outer barrel 20, so that the inner barrel 30 can make a relatively large range of movement relative to the outer barrel 20.

The plunger 40 has a push top 41 at a front end thereof and a thumb rest 42 at an opposing rear end thereof. The plunger 40 is inserted through the second rear end opening 35 of the inner barrel 30 into the inside of the inner barrel 30, so that the push top 41 of the plunger 40 is located in the inner barrel 30. At this time, the thumb rest 42 of the plunger 40 is located outside the inner barrel 30 for a medical staff to press.

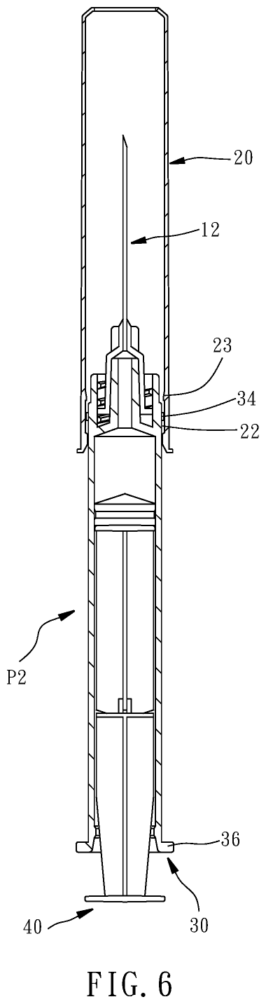

It can be known from the above that the inner barrel 30 is first maintained at the first position P1 and then pushed forward, so that the screwing base 32 of the needle mount 31 of the inner barrel 30 abuts against the periphery of the front end opening 24 of the outer barrel 20, as shown in FIG. 5. At this time, the adapter shaft 33 of the needle mount 31 of the inner barrel 30 will be partially exposed to the outside of the outer barrel 20 through the front end opening 24 of the outer barrel 20, so that the needle 12 is exposed outside the outer barrel 20. Then press the thumb rest 42 of the plunger 40, so that the push top 41 of the plunger 40 is forced to push the medicinal solution out of the inner barrel 30 through the needle 12 for injection. After the injection is completed, pull the finger flange 36 of the inner barrel 30 to make the inner barrel 30, along with the needle 12 and the plunger 40 move backward toward the second position P2. Since the inner diameter of the second annular flange 23 of the outer barrel 20 gradually increases in direction away from the receiving groove 22 of the outer barrel 20 and the protruding height of the positioning portion 34 of the inner barrel 30 is limited, it is not necessary to apply a large amount of pull force to the inner barrel 30 to move the positioning portion 34 of the inner barrel 30 over the second annular flange 23 of the outer barrel 20 to the receiving groove 22 of the outer barrel 20, enabling the inner barrel 30 to be located at the second position P2, as shown in FIG. 6. At this time, the needle 12 is kept inside the outer barrel 20 to increase safety after the injection is completed.

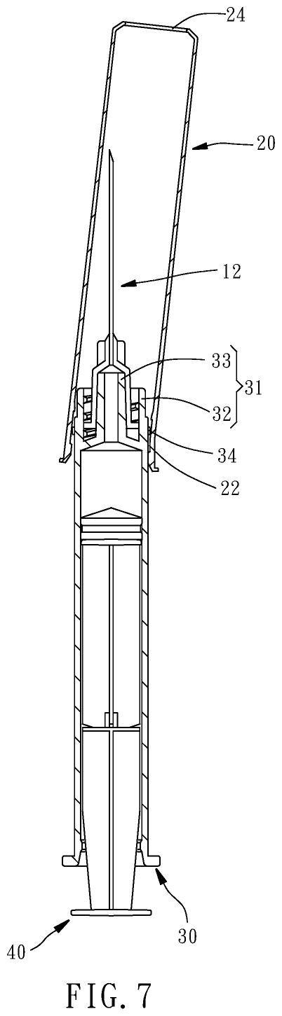

On the other hand, when the inner barrel 30 is located at the second position P2 as shown in FIG. 6, based on the material characteristics of the outer barrel 20, plus the relationship that the receiving groove 22 of the outer barrel 20 is larger in axial length than the positioning portion 34 of the inner barrel 30, at this time, the outer barrel 20 can be further bent to skew relative to the inner barrel 30, as shown in FIG. 7.

The main thing is to make the outer barrel 20 skew so that the front end opening 24 of the outer barrel 20 and the needle mount 31 of the inner barrel 30 are staggered from each other and not on the same line. In this case, even the inner barrel 30 or the plunger 40 accidental collision, the needle 12 will not extend through the front end opening 24 of the outer barrel 20 to the outside of the outer barrel 20, to further ensure that the needle 12 has sufficient safety after injection.

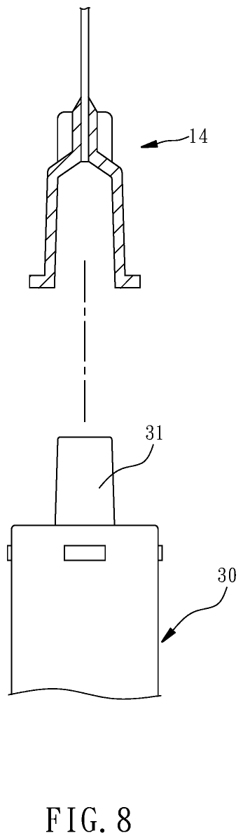

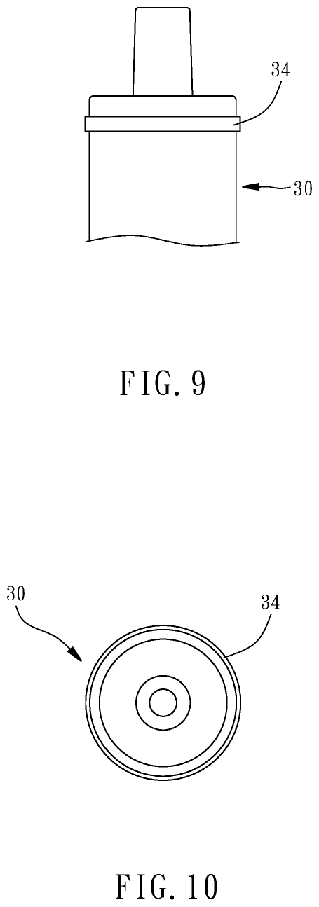

It should be added here that in order to cooperate with different forms of the needle 14, the needle mount 31 of the inner barrel 30 can have different structural changes. As shown in FIG. 8, in the second embodiment of the present invention, the needle mount 31 is a single adapter shaft, and the needle 14 is assembled by directly attaching onto the single adapter shaft. In addition, as shown in FIGS. 9 and 10, the positioning portion 34 of the inner barrel 30 is not necessarily formed by four bumps. In the third embodiment of the present invention, the positioning portion 34 of the inner barrel 30 is a single annular bump, so that is can also achieve a snap-in effect with the first annular flange 21 and second annular flange 23 of the outer barrel 20.

In conclusion, the safety syringe 10 of the present invention uses the cooperation between the positioning portion 34 of the inner barrel 30 and the receiving groove 22 of the outer barrel 20, plus the position-limiting effect provided by the first annular flange 21 and second annular flange 23 of the outer barrel 20 to effectively simplify the structure compared to the prior art, and the overall assembly of the safety syringe 10 can be completed without much effort, thereby achieving the object of the present invention.

Of course, the present invention may have many other embodiments. Without departing from the spirit and essence of the present invention, those skilled in the art can make various corresponding changes and modifications according to the present invention, but these corresponding changes and modifications should fall within the protection scope of the claims attached to the present invention.

What is claimed is:

1. A safety syringe, comprising:
an outer barrel comprising a front end opening located in a front end thereof, a first rear end opening located in an opposing rear end thereof, a first annular flange and a second annular flange located on an inner surface of the rear end of said outer barrel, and a receiving groove located on the inner surface of the rear end of said outer barrel between said first annular flange and said second annular flange;
an inner barrel comprising a needle mount located on a front end thereof, a positioning portion adjacent to said needle mount, a second rear end opening located in an opposing rear end thereof; and
a plunger inserted through said second rear end opening of said inner barrel into the inside of said inner barrel and axially movable relative to said inner barrel;
wherein said inner barrel is inserted through said first rear end opening of said outer barrel into the inside of said outer barrel and axially movable relative to said outer barrel between a first position where said positioning portion of said inner barrel is located between said front end opening of said outer barrel and said second annular flange of said outer barrel so that said needle mount of said inner barrel is partially exposed to the outside of said outer barrel through said front end opening and a second position where said positioning portion of said inner barrel is located in said receiving groove of said outer barrel and blocked by said first annular flange and said second annular flange of said outer barrel so that said needle mount of said inner barrel is completely inside said outer barrel.

2. The safety syringe as claimed in claim 1, wherein said first rear end opening of said outer barrel is adjacent to said first annular flange of said outer barrel, and the inner diameter of said first rear end opening of said outer barrel gradually increases in direction away from said first annular flange of said outer barrel.

3. The safety syringe as claimed in claim 1, wherein the inner diameter of said second annular flange of said outer barrel gradually increases in direction away from said receiving groove of said outer barrel.

4. The safety syringe as claimed in claim 1, wherein said positioning portion of said inner barrel is composed of four bumps equiangularly spaced around the central axis of said inner barrel.

5. The safety syringe as claimed in claim 1, wherein said positioning portion of said inner barrel is an annular bump.

6. The safety syringe as claimed in claim 1, wherein the axial length of said receiving groove of said outer barrel is larger than the axial length of said positioning portion of said inner barrel, so that when said inner barrel is at said second position, said outer barrel is bendable relative to said inner barrel, making said front end opening of said outer barrel and said needle mount of said inner barrel staggered from each other without being on the same line.
